(19) United States
(12) Patent Application Publication
(10) Pub. No.: US 2004/0061606 A1
(43) Pub. Date: Apr. 1, 2004

(54) System and method for surveillance of animals

(76) Inventor: Alexander Gronvold, Vegarshei (NO)

Correspondence Address: YOUNG & THOMPSON, 745 SOUTH 23RD STREET 2ND FLOOR, ARLINGTON, VA, 22202

(21) Appl. No.: 10/416,342

(22) PCT Filed: Nov. 13, 2001
(86) PCT No.: PCT/NO01/00451

(30) Foreign Application Priority Data
Nov. 13, 2000 (NO) 20005721

(51) Int. Cl. G08B 1/08

(52) U.S. Cl. 340/539.11; 340/573.1

(57) ABSTRACT

A system for predator protection and health monitoring of animals. A collar (100) is fastened around the neck of a first animal (A). The collar compromises an electronic unit (200), a communication unit (220) for activation and reception of signals from a passive prone (300) implanted in the animal's body, means (250) for two-way communication with a peripheral unit (400), a GPS unit (260) for communication with a peripheral GPS unit (265), means for registering audible signals, and means for transmitting selected audible signals. The probe (300) registers one or more of any physiological parameter in the animal and communicates these parameters to said communication unit (220). An alarm is given if one physiological parameter differs from predefined values. The alarm may be a pre-programmed audible signal in the electronic unit which is delivered with the aid of a loudspeaker (230) installed in the collar (100), and/or an electronic signal that is transmitted to the peripheral communication unit (400). The electronic alarm signal in the peripheral unit (400) can be given in the form of an audible signal, a text message or a combination thereof. In an alternative embodiment, the collar may comprise a unit (700) for communication with a further communication and control unit (710) in a second collar fastened on a second animal (B). In another embodiment, the collar may be a unit that is permanently mounted on a structure in the immediate vicinity of animals that are kept in cages, stalls or pens. The system can be used for selective monitoring of the health status of a plurality of animals and for selective protection of a plurality of animals against extenal dangers such as predators. The system can be used for the monitoring and surveillance of any animal, including human beings.

The present invention relates to the monitoring of living organisms, and, more particularly, to a system and method for predator protection and health monitoring of animals by using a registering device (e.g., a collar) mounted on or near an [animal] and where the registering device comprises an electronic unit.

The recent serious cases of predator attacks on domestic animals suggest that the time is past when domestic animals could be put out to summer pasture without any form of monitoring or surveillance. The Storting (Norwegian parliament) has decided that the major predators should have a natural place among Norwegian fauna. This means that attacks on domestic animals put out to pasture will increase in the coming years. Attempts have been made to restrict the area in which the predators can form family groups, but it must be expected that roving animals from these animal populations will be active and predictable predators over a considerably larger area.

It is obvious that the patrolling of these areas by herdsmen would be financially unacceptable. However, the use of an electronic alarm would allow the herdsman function to be performed when and where an alarm goes off. The herdsman function could then in most cases be carried out by the owner of the animals, and without any major costs.

Today there are about 2.4 million sheep in Norway. Almost all these animals roam freely in open country (rough grazing) for large parts of the year. About 130,000 sheep disappear each year when put out to pasture. Of these, it is estimated that about 33,000 are killed by predators, and this results in large insurance payouts.

Various electronic systems for monitoring (tending) animals are known. For example, U.S. Pat. No. 5,818,354 (“Animal Monitoring System”, Gentry, 06.10.98) describes a collar that can be placed around the animal's neck. The collar is equipped with two sensors which measure the animal's heart rate and temperature. Furthermore, the collar comprises an encoder and a radio transmitter which transmits the signals to a receiver that is carried by, e.g., the animal's owner. The system described in the aforementioned document permits the monitoring of one animal at a time. This collar is considered to be unreliable, mainly because the sensors will not have a stable contact surface with the animal's skin when the animal moves. The sensors will also be easily affected by the ambient temperature. Moreover, the collar is considered to be very uncomfortable for the animal and probably not legal for use on livestock that roam free.

Furthermore, U.S. Pat. No. 4,876,674 (“Sonic Collar Sheep Protector”, Parmely et al., 24.10.89) comprises a collar that is placed around the neck of a sheep. The collar comprises means which detect rapid and sudden movement which occurs, e.g., when the sheep is frightened, and converts the movements into electrical signals. These signals then generate a pre-programmed sound and/or light signal which is intended to scare away any predators. A similar “talking sheep bell” is also known to have been used in Norway. Some of the drawbacks of these pre-programmed units are that they cannot distinguish between real situations of danger and, e.g., instances when the sheep moves quickly and suddenly for other reasons. Furthermore, an intelligent predator, as for instance a wolf, can soon become accustomed to the alarm and learn that it does not constitute a threat. Moreover, this collar does not alert the farmer in any other way than via sound signals. This collar is therefore not considered to be essentially different from ordinary sheep bells.

Accordingly, there is believed to be a long-felt want for a flexible, reliable and inexpensive system for monitoring animals, primarily a system for predator protection and health monitoring of animals put out to pasture, and where a system of this kind does not cause any discomfort or distress to the animal.

Therefore, according to the invention, there is provided a system of the aforementioned type and as disclosed in the preamble of the attached claims. The system is thus characterised in that it comprises:

a communication unit for activation and reception of signals from a passive probe implanted in the animal's body;

means for two-way communication with a peripheral unit;

a GPS unit for communication with a peripheral GPS unit;

means for registering audible signals; and

means for transmitting selected audible signals.

Preferred features of the system according to the invention are set forth in attached claims 2-20.

According to the invention, there is also provided a method which is characterised in that:

a communication unit installed in a collar selectively activates a passive probe implanted in the animal's body, and receives signals concerning the animal's physiological parameters from said probe;

an alarm is given if one physiological parameter deviates from pre-defined values;

the collar, with the aid of means, is in two-way communication with a peripheral unit;

the collar communicates via a GPS unit with a peripheral GPS unit;

the collar can register audible signals; and

the collar can transmit selected audible signals.

The system according to the invention comprises an electronic collar, primarily intended for domestic animals, which contains an alarm unit with a passive probe for implantation in the animal, a mobile telephone unit, a loudspeaker unit and a GPS unit.

The collar consists of several components assembled to provide an alarm unit. A registering device having a passive probe implanted in the animal. The probe (the sensor) measures/registers one or more physiological functions (e.g., heart rate, body temperature). The registering device reads the reactions or frequencies that are generated, and issues an alarm when they are over a certain predetermined level.

The alarm is transmitted to a mobile telephone unit which calls up or issues an alarm to the animal owner over the NMT 450 or GSM network.

A loudspeaker which can be activated either by the alarm or by the animal owner, and which can deliver a signal that scares off a predator. This signal can be preprogrammed or issued by the animal owner via the telephone, and will be sufficient to cause the predator to interrupt its attack, and thus gives the herdsman a chance to reach the animal before the next attack.

It is of crucial importance that the predator sees that people come to the place in question after the scare signal. Predators are intelligent animals that are not easily tricked when it comes to basic matters such as food. If the scare signal sounds without a herdsman coming on the scene, the predator will quickly understand this, and the signal will lose its effect. In that case, the only achievement would be that the domestic animal has learnt to wear an electronic device around its neck, whilst the predator has learnt to interpret non-dangerous scare signals. Predators fear human beings, so it is essential that they are confronted with the presence of humans in order to understand that the scare signal really does mean danger.

All the components of the collar are based on available technology. The assembly of the components and their size and shape must be adapted to the animal in question. Materials selection must be made with care when it comes to water-tightness, shock resistance etc. The collar battery pack must be designed having in mind that it should be as small as possible; the battery type will depend on factors such as the power requirement of the components.

Compared with the prior art, the system according to the invention allows an animal owner to continuously monitor any number of animals. The animal owner can listen to the environment around the sheep whenever he wants with the aid of a microphone in the collar. This may be useful in connection with exposing the theft of sheep. Furthermore, the telephone unit allows the animal owner himself to transmit sound signals to a predator which might be in the vicinity of the sheep. As mentioned, predators react to human voices, so this is an effective way of scaring the predator.

The system according to the invention can also be used to monitor the animal's general state of health. If, e.g., heart rate, temperature or other measurable physiological parameters rise above or fall below given limits, an alarm is given in the animal owner's receiver.

Furthermore, the system according to the invention can with the aid of the incorporated GPS unit and, e.g., together with an electronic map in the animal owner's computer, allow an exact geographical localisation of the animal.

Embodiments of the invention are set forth in the attached patent claims, and the following description with reference to the attached drawings. It should be understood that the drawings only show typical exemplary embodiments, and should not be understood as defining the limits of the invention. In the drawings the parts are indicated by reference numerals which will also be used in the following.

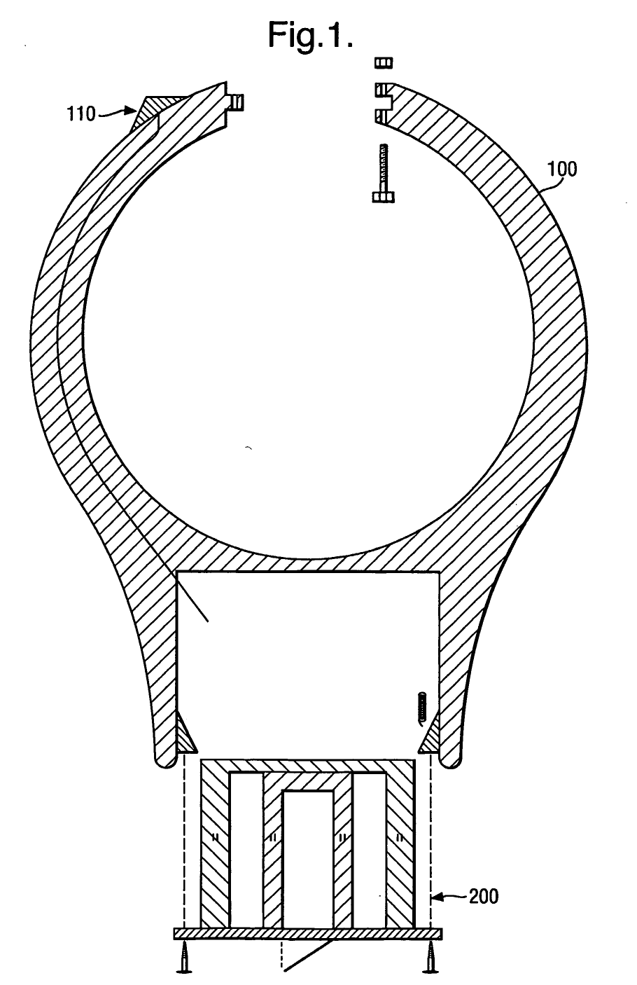

FIG. 1 is a front view of the system according to the invention.

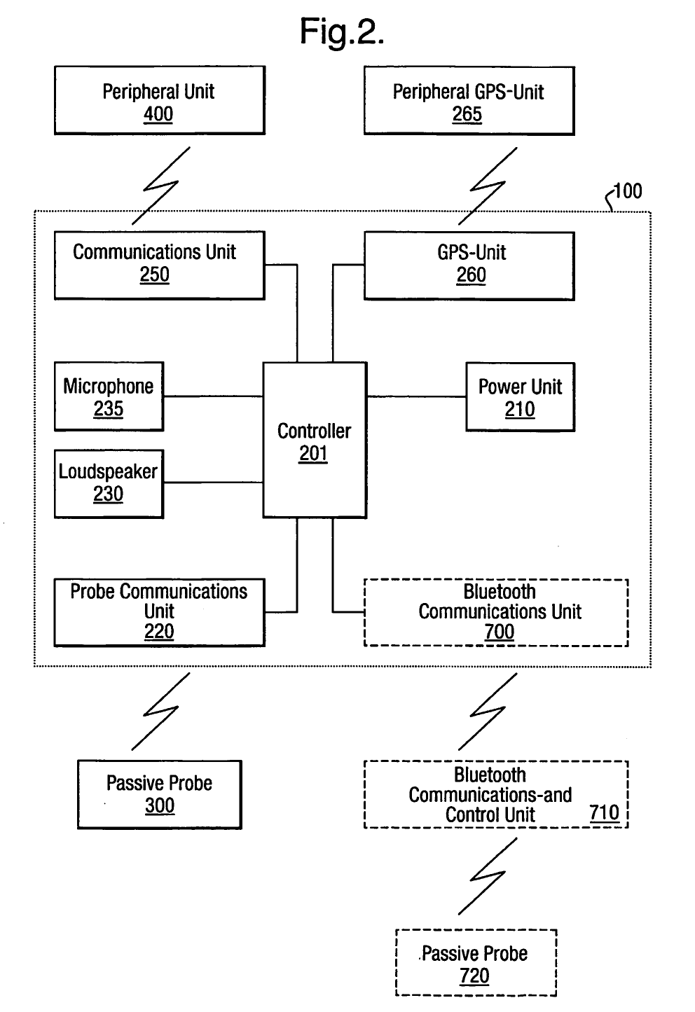

FIG. 2 is a schematic diagram of the system according to the invention.

The system for predator protection and health monitoring of animals comprises, in one exemplary embodiment, a collar 100 which with the aid of fastening means 122 is releasably fastened around the neck of a first animal A. This first animal A may, e.g., be an adult sheep. The collar comprises an electronic unit 200 and a communication unit 220 for activating and receiving signals from a passive probe 300 implanted in the animal's body. The system also comprises means 250 that are known per se for two-way communication with a peripheral unit 400, a GPS unit 260 for communication with a peripheral GPS unit 265, means for registering audible signals, and means for transmitting selected audible signals.

When the passive probe 300 is called up by the communication unit 220, it registers one or more of any of the animal's physiological parameters (e.g., heart rhythm, body temperature) and communicates these parameters to the communication unit 220.

If one physiological parameter differs from predetermined values, an alarm is transmitted via, e.g., the GPS unit to the peripheral units.

As mentioned, the alarm in the collar can be a pre-programmed audible signal that is delivered via a loudspeaker 230 installed in the collar 100, and/or the alarm can be an electronic signal that is transmitted to the peripheral communication unit 400. The electronic alarm signal in the peripheral unit 400 can be given as an audible signal, a text message or a combination thereof. In one exemplary embodiment, the means 250 for two-way communication and the peripheral unit 400 are mobile telephone units.

The audible signals close to the animal can be registered by means of a microphone 235 installed in the collar 100 and transmitted for reproduction in the peripheral communication unit 400.

Furthermore, by using the peripheral communication unit 400, a person can deliver selected audible signals with the aid of a loudspeaker 230 installed in the collar 100.

The electronic unit 200 also contains a unique identity code which is transmittable and readable in the peripheral communication unit 400. Furthermore, the electronic unit 200 can be operated by means of the peripheral communication unit 400.

If the battery voltage of the electronic unit falls below a pre-set level, the electronic unit 200 transmits a message to the peripheral communication unit 400.

As mentioned, the collar 100 is equipped with a GPS unit 260 for communication of the animal's position to a peripheral GPS unit 265. The peripheral GPS unit 265 comprises a GPS receiver which may be integrated with an electronic map, e.g., in a PC.

In an alternative embodiment, the collar 100, which is fitted on a first animal A (e.g., one of a plurality of adult animals, such a ewe), can comprise a unit 700 which communicates with one or more additional communication and control units 710 in other collars on one or more other animals B (e.g., young animals such as lambs). The communication and control unit 710 in this (or these) other collar(s) collects physiological parameters from the respective other animal(s) in a similar way. These physiological parameters from the other animal(s) are communicated to the peripheral unit 400 via the communication unit in the first animal's collar. As mentioned, the peripheral units 400, 265 can communicate with several collars that contain respective components as mentioned above.

Thus, through the use of the system described above, animals can be monitored by means of a method wherein:

a communication unit 220 fitted in the collar selectively activates a passive probe 300 implanted in the animal's body, and receives signals concerning the animal's physiological parameters from said probe;

an alarm is given if one physiological parameter differs from predetermined values;

the collar, with the aid of means 250, is in two-way communication with a peripheral unit 400;

the collar with the aid of a GPS unit 260 communicates with a peripheral GPS unit 265;

the collar can selectively register audible signals; and

the collar can selectively transmit selected audible signals.

As mentioned, the system according to the invention can be used for selective monitoring of the health status of a plurality of animals. The system according to the invention can selectively be used for selective protection of a plurality of animals from external dangers such as predators. The system can also be used for the surveillance of several animals.

In an alternative embodiment, the system according to the invention can be used for the monitoring and surveillance of any animal or group of animals. The system can also be used to check and monitor, e.g., humans suffering from physical disorders (patients) who can thus move freely around in a specific area (e.g., a nursing institution). The system can also be used to check the movements of people serving a sentence, where these persons serve their sentence at liberty and can move within a pre-agreed area (e.g., within a city boundary).

Furthermore, in an alternative embodiment, the system can be used to monitor animals in stables or barns, where the collar is replaced by a basically similar unit which is fixedly mounted (e.g., in the ceiling) in the vicinity of the animal or animals that are to be monitored. This may be a suitable embodiment for monitoring, e.g., valuable animals such as racehorses, or other animals in connection with, e.g., disease or an imminent birth.

1. A system for predator protection and health monitoring of animals, preferably livestock, where a device (100) is releasably fitted on a first animal (A) and where the device consists of an electronic unit (200), means (250) for two-way communication with a peripheral unit (400), a GPS unit (260) for communication with a peripheral GPS unit (265),
characterised in that the system comprises:
a communication unit (220) in the device (100) for wireless activation and reception of signals from a passive probe (300) implanted in the animal's body;
means (235) in the device (100) for registering audible signals; and
means (230) in the device (100) for transmitting selected audible signals,
wherein the probe (300), when it is called up by the communication unit (220), registers one or more of any measurable physiological parameters in the animal and communicates these parameters to the communication unit (220) in the device (100) for transmission to a peripheral unit, where said peripheral unit is adapted to issue an alarm if one physiological parameter differs from predefined values.

2. A system according to claim 1, characterised in the electronic alarm signal in the peripheral unit (400) is given in the form of an audible signal, a text message or a combination thereof.

3. A system according to claim 1, characterised in that the means (250) for two-way communication and the peripheral unit (400) are mobile telephone units.

4. A system according to claims 1 and 2, characterised in that said audible signals are registered by means of a microphone (235) installed in the device (100) and are transmitted for reproduction in the peripheral communication unit (400).

5. A system according to claim 1, characterised in that a person by using the peripheral communication unit (400) can deliver selected audible signals with the aid of a loudspeaker (230) installed in the device (100).

6. A system according to claim 1, characterised in that the electronic unit (200) contains a unique identity code which is transmittable and readable in the peripheral communication unit (400).

7. A system according to claim 1, characterised in that the electronic unit (200) can be operated by means of the peripheral communication unit (400).

8. A system according to claim 1, characterised in that the electronic unit (200) transmits a message to the peripheral communication unit if the battery voltage of the electronic unit falls below a pre-set level.

9. A system according to claim 1, characterised in that the device (100) is equipped with a GPS unit (260) for communication of the animal's position to a peripheral GPS unit (265).

10. A system according to claims 1 and 9, characterised in that said peripheral GPS unit (265) comprises a GPS receiver integrated with an electronic map in a PC.

11. A system according to claim 1, characterised in that the device (100) fitted on said first animal (A) comprises a unit (700) for communication with a further communication and control unit (710) in a second device releasably fitted on a second animal (B).

12. A system according to claim 11, characterised in that said communication and control unit (710) in said device on said second animal (B) collects physiological parameters from said second animal by using a passive probe (220) implanted in said second animal.

13. A system according to claims 1 and 12, characterised in that said physiological parameters from said second animal are communicated to the peripheral unit (400) via said communication unit in the first animal's (A) device (100).

14. A system according to any one of claims 1-13, characterised in that the peripheral units (400, 265) can communicate with a plurality of devices containing respective components as disclosed in the preceding claims.

15. A system according to any one of claims 1-14, characterised in that the device (100) is a unit which is permanently mounted on a structure in the immediate vicinity of animals kept in cages, stalls or pens.

16. A system according to any one of claims 1-14, characterised in that the device (100) is a collar releasably fitted around the animal's neck.

17. A method for monitoring animals, wherein a device (100) is releasably fitted on a first animal (A), and the device, with the aid of means (250), is in two-way communication with a peripheral unit (400), and the device communicates via a GPS unit (260) with a peripheral GPS unit (265),
characterised in that
a communication unit (220) installed in the collar selectively activates a passive probe (300) implanted in the animal's body, and receives signals concerning the animal's physiological parameters from said probe;
an alarm is issued if one physiological parameter differs from predetermined values;
the collar can selectively register audible signals; and
the collar can selectively transmit selected audible signals,
wherein the probe (300), when it is called up by the communication unit (220), registers one or more of any measurable physiological parameters in the animal and communicates these parameters to said communication unit (220), where by means of the said system an alarm is issued if one physiological parameter differs from predefined values.

18. A method according to claim 17, characterised in that the device (100) is a unit that is permanently mounted on a structure in the immediate vicinity of animals that are kept in cages, stalls or pens.

19. A method according to claims 17 and 18, characterised in that it is performed by means of the components as disclosed in claims 1 to 21.

20. A method according to claim 17, characterised in that the device (100) is a collar capable of being releasably fitted around the animal's neck.

21. The use of the system as disclosed in claims 1 to 16, wherein the first animal (A) is one of a plurality of adult animals and the second animal (B) is one of a plurality of young animals.

22. The use of the system as disclosed in claims 1 to 16 for selective monitoring of the state of health of a plurality of animals.

23. The use of the system as disclosed in claims 1 to 16 for selective protection of a plurality of animals against external dangers such as predators.

24. The use of the system as disclosed in claims 1 to 16 for selective supervision of a plurality of animals.

25. The use of the system as disclosed in claims 1 to 16 on a plurality of any animal, including human beings.
